(19) United States
(12) Patent Application Publication
(10) Pub. No.: US 2022/0268767 A1
(43) Pub. Date: Aug. 25, 2022

(54) NANOPARTICLES FOR DETECTION OF BACTERIA CELLS AND METHODS OF PREPARATION THEREOF

(71) Applicant: AGENCY FOR SCIENCE, TECHNOLOGY AND RESEARCH, Singapore (SG)

(72) Inventors: Xinting ZHENG, Singapore (SG); Yen Nee TAN, Singapore (SG); Choon Peng TENG, Singapore (SG); Benedict You Wei HSU, Singapore (SG); Takuya SATO, Singapore (SG)

(21) Appl. No.: 17/639,469

(22) PCT Filed: Sep. 1, 2020
(86) PCT No.: PCT/SG2020/050507

§ 371 Date: Mar. 1, 2022

(30) Foreign Application Priority Data
Sep. 4, 2019 (SG) 10201908147X

(51) Int. Cl. G01N 33/543; C08G 73/02

(52) U.S. Cl. CPC G01N 33/54346; C08G 73/02; B82Y 30/00

(57) ABSTRACT

The present disclosure relates, in general terms, to nanoparticles which are characterised by having a photoluminescent amorphous core and a surface is functionalised with at least a carbohydrate. The present disclosure also relates to methods of forming and functionalising the nanoparticles and a photoluminescence assay comprising the nanoparticles for quantifying a sample comprising bacterial cells.

### TECHNICAL FIELD

The present disclosure relates, in general terms, to nanoparticles which are characterised by having a photoluminescent amorphous core and a surface is functionalised with at least a carbohydrate. The present disclosure also relates to methods of forming and functionalising the nanoparticles and a photoluminescence assay comprising the nanoparticles for quantifying a sample comprising bacterial cells.

### BACKGROUND

Microbial pathogens pose serious threats to public health and safety, and result in millions of illnesses and deaths as well as huge economic losses annually. Bacterial infectious diseases cause approximately one third of all global deaths. In addition, according to The U.S. Department of Agriculture's Economic Research Service, the cost of foodborne-illness is estimated to be 15.6 billion dollars annually. To date, many foodborne illnesses caused by pathogenic bacteria such as Escherichia coli O157:H7, Staphylococcus aureus, and Salmonella species have been reported. As these pathogenic bacteria can cause severe symptoms such as diarrhoea, vomiting etc. and even result in fatality, there is a need to develop rapid on-site detection methods.

A rapid detection method for pathogenic bacteria with high sensitivity for detecting very low bacteria concentrations is very critical to assure food safety. Conventional bacteria detection methods, such as the plating technique and biochemical methods, are reliable, highly robust, and accurate. However, they are not particularly suited to be used as user-friendly approaches since they require time consuming culture processes (up to 3 days), trained operators and relatively sophisticated laboratory equipment. Furthermore, these methods require at least 18 h from sampling to results and this long delay to identify the pathogenic bacteria can be disastrous due to the loss of valuable time for treating the disease. Thus, there is an urgent need to develop rapid and reliable methods to detect bacteria in environmental, food, and water matrices. Although there are faster detection methods such as polymerase chain reaction (PCR), immunoassays and magnetic force based methods, they require the use of external equipment and complicated operation principle which make on-site detection difficult. As such, current laborious and expensive pathogen tests often represent a significant hindrance to implementing effective front-line preventative care.

Thus, there is a significant need to develop low-cost and easy-to-use methods for microbial pathogen detection.

It would be desirable to overcome or ameliorate at least one of the above-described problems, or at least to provide a useful alternative.

### SUMMARY

The present invention is predicated on photoluminescent nanodots (or nanoparticles) which are derived from biomolecules such as amino acids (e.g. Thr) and carbohydrates. Advantageously, these carbohydrate functionalised nanoparticles exhibit high affinity towards certain bacteria strains (e.g. mannose that binds E.coli) compared to unfunctionalised nanoparticles. The nanoparticles can be formed using a polymeric precursor polyethylenimine (PEI). These nanoparticles exhibit bright fluorescence and can be used for quantitative fluorescent detection (sensitive detection and high affinity moiety) of target specific bacterial cells. It is also found to exhibit effective bacteria inhibition capability. The method of preparing the nanoparticles involves a two-step one-pot hydrothermal process. For target specific bacteria detection, silica coated magnetic particles were further developed to minimise fluorescence quenching to these nanoparticles. The magnetic particles can be conjugated with bacteria-specific antibodies to enable fast and effective magnetic capture (˜100% capture efficiency), separation and concentration of target bacteria. Combining the above mentioned multifunctional nanoparticle reagents, a 2 step culture-free fluorescent method for quantitative bacteria detection with a limit of detection (LOD) of 10⁴ CFU/mL is exemplified.

The present invention relates to a nanoparticle which is characterised by a photoluminescent core having a surface,

wherein the core is an amorphous core formed from threonine and polyethylenimine (PEI); and

wherein the surface is functionalised with at least a carbohydrate.

In some embodiments, the photoluminescence from the core is emittable within the range of about 400 nm to about 650 nm.

In some embodiments, the emittable photoluminescence is at least 1.5 times a comparator nanoparticle which is not formed from both threonine and PEI.

In some embodiments, the comparator is selected from the group consisting of:

i) a nanoparticle having a core formed from serine or threonine or PEI;

ii) a nanoparticle having a core formed from serine and PEI;

iii) a nanoparticle having a core formed from threonine and chitosan;

iv) a nanoparticle having a core formed from serine and dextran (DEX);

v) a nanoparticle having a core formed from serine and hyaluronic acid (HA);

vi) a nanoparticle having a core formed from serine and polyethylene glycol monomethyl ether (mPEG); and

v) a nanoparticle having a core formed from serine and poly(L-lysine) (PLL).

In some embodiments, the PEI is a branched PEI.

In some embodiments, the core has a molecular weight of less than 3 kD.

In some embodiments, the carbohydrate is mannose.

In some embodiments, the nanoparticle has a mean diameter of about 1 nm to about 8 nm.

In some embodiments, the nanoparticle has a photoluminescence stability of at least 95% after 30 min irradiation.

In some embodiments, the photoluminescence is fluorescence.

In some embodiments, the nanoparticle has a zeta potential of more than about +5 mV.

In some embodiments, the nanoparticle has a minimum inhibitory concentration (MIC) value against bacteria of more than 150 μg/mL.

The present invention also relates to a method of forming and functionalising a nanoparticle, including the steps of:

a) hydrothermally reacting threonine and PEI to form the nanoparticle having a photoluminescent amorphous core; and

b) reacting at least a carbohydrate with a surface of the nanoparticle core in order to form a surface functionalised nanoparticle with at least a carbohydrate.

In some embodiments, step (a) is performed at high temperature and pressure.

In some embodiments, step (b) is performed at about 60 ° C. for about 48 h.

In some embodiments, the method further includes a step of filtrating the core from the unreacted threonine and PEI after step (a).

In some embodiments, the method further includes a step of purifying the nanoparticle after step (b).

The present invention also relates to a photoluminescence assay for quantifying a sample comprising bacterial cells, including the steps of:

a) incubating the sample with a silica coated magnetic particle for allowing the silica coated magnetic particle to contact the bacterial cells;

b) exposing the bacterial cells contacted with the silica coated magnetic particle to an external magnetic field to form a first magnetic pellet and a first supernatant;

c) separating the first magnetic pellet from the first supernatant;

d) incubating the first magnetic pellet with a nanoparticle for allowing the nanoparticle to contact the bacterial cells, the nanoparticle is characterised by a photoluminescent amorphous core having a surface, wherein the core is formed from threonine and polyethylenimine (PEI), and wherein the surface is functionalised with at least a carbohydrate;

e) exposing the bacterial cells contacted with the silica coated magnetic particle and the nanoparticle to an external magnetic field to form a second magnetic pellet and a second supernatant; and

f) quantifying the emittable photoluminescence from the second magnetic pellet.

In some embodiments, step (a) is performed for at least 10 min.

In some embodiments, the silica coated magnetic particle has a mean particle size of about 15 nm to about 40 nm.

In some embodiments, the silica coated magnetic particle has a silica shell thickness of at least about 5 nm.

In some embodiments, the silica coated magnetic particle has a magnetic particle core mean particle size of about 10 nm.

In some embodiments, the silica coated magnetic particle has a saturation magnetization of more than about 40 emu/g.

In some embodiments, the silica coated magnetic particle has negligible remanence.

In some embodiments, the silica coated magnetic particle has a zeta potential of about −5 mV to about +5 mV.

In some embodiments, the silica coated magnetic particle is at least partially functionalised with an amino moiety.

In some embodiments, the silica coated magnetic particle is conjugated to an antibody for targeting bacterial cells.

In some embodiments, the assay further includes a step of resuspending the second magnetic pellet in a solvent after step (e).

In some embodiments, the assay further includes a step of comparing the emitted photoluminescence with a calibration plot for determining the concentration of bacterial cells in the sample after step (f).

In some embodiments, the assay has a detection limit of at least 10⁴ CFU/mL of bacterial cells.

### BRIEF DESCRIPTION OF THE DRAWINGS

Embodiments of the present invention will now be described, by way of non-limiting example, with reference to the drawings in which:

FIG. 1 is a comparison of three generations of biodots (nanoparticles) for fluorescence bacteria labelling; the abbreviations are as follows: serine biodots (SD), threonine biodots (TD), PEI biodots (PD), serine-PEI biodots (SP), threonine-PEI biodots (TP), serine-chitosan biodots (SC), threonine-chitosan biodots (TC), serine-mPEG biodots (S-mPEG), serine-dextran biodots (S-DEX), serine-hyaluronic acid biodots (S-HA), serine-poly(L-lysine) biodots (S-PLL), threonine-PEI-mannose biodots (TPM);

FIG. 2 shows (A) synthesis route for TPMdot; (B) UV-vis absorption spectra of SPdot, TPdot, TPMdot and mannose (Inset shows the photographs of the dots under white light and UV irradiation); (C) photoluminescence spectra of SPdot, TPdot, TPMdot; (D) TEM image of TPMdots; (E) zeta potential values of SPdot, TPdot, TPMdot; (F) excitation dependent PL property of TPMdots and (G) photostability study of SPdot, TPdot, TPMdot and FITC;

FIG. 3 shows bacterial inhibition study for (A) E.coli and (B) S. aureus treated with SP, TP or TPM dots;

FIG. 4 shows (A) TEM image of magnetic particles (MPs) after silica encapsulation; (B) Field-dependent magnetization curve of magnetic particles at room temperature; (C) Zeta potential measurements of MPs with varying molar ratio of TEOS/APTES; (D) Silica-coated magnetic particles (IMRE MP) show negligible quenching of biodots;

FIG. 5 shows (A) Schematic of strategy to prepare conjugated magnetic particles based on carbodiimide chemistry; (B) Graph of optical density (OD) readings indicating the capture of E.coli by magnetic particles of different zeta potential;

FIG. 6 shows schematic of bacteria capture and separation with photos of plate counting results. 1 mg of conjugated magnetic particles (carbodiimide method) captures ˜1.10 ×10⁵ CFU/mL of E.coli (average of 2 batches);

FIG. 7 shows (A) Experimental steps depicting the fluorescence assay for E.coli quantification (B) Photoluminescence readings of biodots uptaken by E.coli after capture by MPs and reproducibility of detection results (C) Calibration curve of PL against E.coli concentration; and

FIG. 8 shows a exemplary Fast Fourier Transform (FFT) pattern of TPMdots.

### DETAILED DESCRIPTION

The present invention is predicated on the discovery that certain nanoparticles are photoluminescent. However, to be suitable for use in a bacteria assay, other requirements must also be met.

Towards this end, the inventors have developed biodots (or nanoparticles) derived from biomolecules including threonine, mannose and PEI polymer in a simple two-steps hydrothermal process that exhibit multiple functions:

1. High brightness for fluorescence labelling

2. Excellent photo-stability for accurate fluorescence sensing

3. Ultra-small size and positive surface charge for fast bacteria interaction and uptake

4. Surface affinity moieties for specific targeting to a particular bacteria cell type

Accordingly, the present invention relates to a nanoparticle which is characterised by a photoluminescent core having a surface,

wherein the core is an amorphous core formed from threonine and polyethylenimine (PEI); and

wherein the surface is functionalised with at least a carbohydrate.

The inventors have found that a nanoparticle with a core formed from threonine and PEI is particularly advantageous. To this end, compared to other amorphous nanoparticles, the nanoparticles of the present invention particularly has a stronger and more stable photoluminescence. Without wanting to be bound by theory, it is believed that the core formed from a hydrothermal reaction can trigger the precursors (i.e. threonine and PEI) to undergo polymerization or crosslinking. It is believed that the combination of threonine and PEI allows for a further rearrangement and finally aromatize into conjugated pi system. This is advantageous for providing the high and/or stable photoluminescent property of the nanoparticle, in contrast to other amorphous core nanoparticles which can have numerous electron-hole defects which act to reduce the photoluminescent property.

Further, amorphous threonine-PEI nanoparticles are believed to be able to bind to bacteria more efficiently and subsequently being taken up by the cells, so more threonine-PEI dots can bind to the same amount of bacteria thus giving brighter photoluminescence to the stained bacteria cells.

Further, functionalisation with a carbohydrate is particularly advantageous as specific bacterial cells can be targeted when used in an assay. Compared to unfunctionalised nanoparticles, carbohydrate functionalised nanoparticles are able to be endocytosed to a larger extend without causing the bacterial cells to undergo apoptosis. Additionally, unspecific binding is largely reduced. This allows for the bacteria assay to be more accurate and/or precise.

In contrast, crystalline nanoparticles, require extreme synthesis conditions and harsh reagents which are not environmentally friendly. As a result, the average cost of such crystalline nanoparticles tend to be on the high end, and thus are not suitable for use in a versatile and portable fluorescence sensor/assay.

In this regard, such nanoparticles can act as fluorescence probes and allow for easy quantification which represent an attractive option for bacteria sensing. Furthermore, these nanoparticles are also versatile and can be used in commercially available portable fluorescence sensors available in the market to facilitate for on-site detection.

As used herein, nanoparticles or nanodots or biodots refer to particles of matter having any shape with dimensions in the range of about 1×10⁻⁹ m and about 1×10⁻⁷ m. The average particle size can be equivalent to the mean diameter of the nanoparticle. The present definition also includes anisotropic nanoparticles. Such anisotropic nanoparticles can, for example, include non-spherical nanoparticles, nanorods, nanocubes, nanochains, nanostars, nanoflowers, nanoreefs, nanowhiskers, nanofibers, and nanoboxes.

As used herein, “photoluminescence” refers to light emission from any form of matter after the absorption of photons (electromagnetic radiation). It is one of many forms of luminescence (light emission) and is initiated by photoexcitation (i.e. photons that excite electrons to a higher energy level in an atom), hence the prefix photo-. Following excitation various relaxation processes typically occur in which other photons are re-radiated. More processes may occur when a substance undergoes internal energy transitions before re-emitting the energy from the absorption event. Electrons change energy states by either resonantly gaining energy from absorption of a photon or losing energy by emitting photons. One form of photoluminescence is fluorescence. Fluorescence is typically a fast process, yet some amount of the original energy is dissipated so that re-emitted light photons will have lower energy than did the absorbed excitation photons. The re-emitted photon in this case is said to be red shifted, referring to the reduced energy it carries following this loss (as the Jablonski diagram shows).

Another form of photoluminescence is phosphorescence, in which electrons which absorbed photons, undergo intersystem crossing where they enter into a state with altered spin multiplicity (see term symbol), usually a triplet state. Once the excited electron is transferred into this triplet state, electron transition (relaxation) back to the lower singlet state energies is quantum mechanically forbidden, meaning that it happens much more slowly than other transitions. The result is a slow process of radiative transition back to the singlet state, sometimes lasting minutes or hours. This is the basis for “glow in the dark” substances.

Amorphous refers to a non-crystalline solid that lacks the long-range order that is characteristic of a crystal. Amorphous materials have an internal structure made of interconnected structural blocks. These blocks can be similar to the basic structural units found in the corresponding crystalline phase of the same compound. Whether a material is liquid or solid depends primarily on the connectivity between its elementary building blocks so that solids are characterized by a high degree of connectivity whereas structural blocks in fluids have lower connectivity. Amorphous nanoparticles can have some short range order at the atomic length scale due to the nature of chemical bonding. Furthermore, in very small nanoparticles relaxation of the surface and interfacial effects distort the atomic positions, decreasing the structural order.

The skilled person would understand that to characterise amorphous material (as compared to non-amorphous or crystalline material), various techniques such as X-ray absorption spectroscopy (XAS), X-ray diffraction (XRD), inductively coupled plasma (ICP) and high-resolution transmission electron microscopy (HRTEM) can be used. For carbon nanodots, it is more suitable to TEM to characterize.For amorphous carbon nanodots, no discernible lattice structure can be observed in TEM images and the Fast Fourier Transform (FFT) pattern of the sample will show a ring-like electron diffraction pattern.

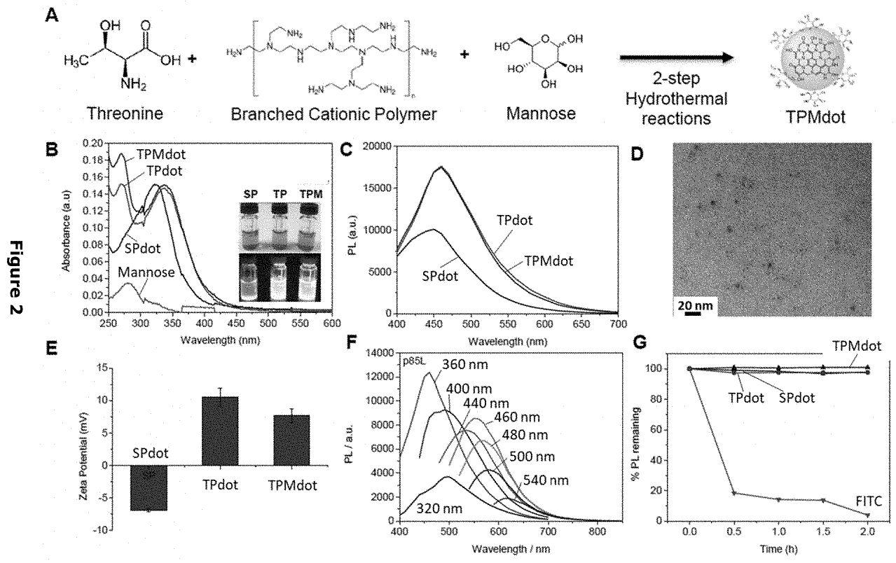

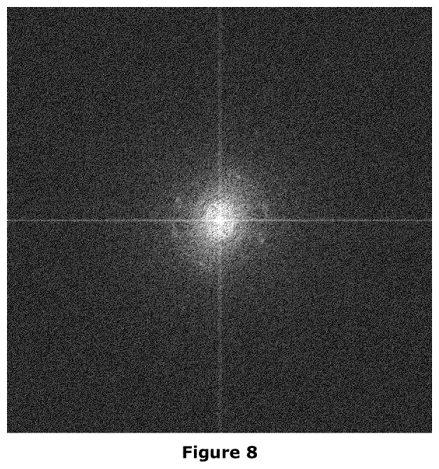

As shown in FIG. 2D, the TEM image of the biodots do not reveal any discernible lattice structures. In addition, the fast fourier transform pattern of the dots shows a ring-like electron-diffraction pattern, indicating the amorphous nature of the sample (FIG. 8).

In some embodiments, the core is formed from threonine and polyethylenimine (PEI). In other embodiments, threonine and polyethylenimine (PEI) are covalently bonded to each other via amide linkages. In some embodiments, the PEI is a branched PEI.

Accordingly, a matrix structure is formed when the branched PEI cross-linked with threonine.

It was found that a branched PEI is particularly advantageous for facilitating formation of covalent bonds with threonine and thus form a more compact nanoparticle core. In this way, the density of crosslinking is increased and this can contribute to a brighter and more stable photoluminescent core.

In some embodiments, the PEI or branched PEI has a molecular weight of about 600. In other embodiments, the molecular weight is about 1,000, about 2,500, about 5,000, about 10,000, about 25,000, about 50,000, about 75,000, about 100,000, about 200,000 or about 300,000. The molecular weight can be determined using analytical methods such as liquid chromatography and/or gel permeation chromatography.

The PEI can have a structure as shown in Formula (II):

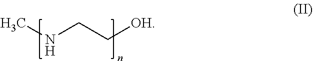

The branched PEI can have a structure as shown in Formula (III):

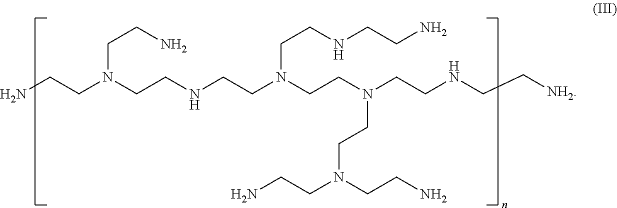

Depending on the molecular weight, the integer n can be determined accordingly.

The PEI and branched PEI (and its salts thereof) as described herein also includes functionalised PEI and functionalised branched PEI. For example, the PEI and branched PEI can be modified with stearic acid, polyethylene glycol (PEG), and hydroxyl group.

In some embodiments, the core has a molecular weight of less than 3 kDa. In other embodiments, the molecular weight is less than 2.5 kDa, less than 2 kDa, less than 1.5 kDa or less than 1 kDa. The molecular weight of the core can be tuned by varying the synthetic conditions. For example, the hydrothermal reaction can be tuned by varying the temperature, pressure and the reaction time.

In some embodiments, the photoluminescence from the core or the nanoparticle is emittable within the range of about 400 nm to about 650 nm. In other embodiments, the photoluminescence is emittable within the range of about 450 nm to about 650 nm, about 500 nm to about 650 nm, about 550 nm to about 650 nm, or about 600 nm to about 650 nm.

In some embodiments, the photoluminescence is fluorescence.

In some embodiments, the emittable photoluminescence is at least 1.5 times a comparator nanoparticle which is not formed from both threonine and PEI. In other embodiments, the emittable photoluminescence is at least 1.4 times, at least 1.3 times, at least 1.2 times, at least 1.1 times, at least 1 time, at least 0.9 times, or at least 0.8 times.

In some embodiments, the comparator is selected from the group consisting of:

i) a nanoparticle having a core formed from serine or threonine or PEI;

ii) a nanoparticle having a core formed from serine and PEI;

iii) a nanoparticle having a core formed from threonine and chitosan;

iv) a nanoparticle having a core formed from serine and dextran (DEX);

v) a nanoparticle having a core formed from serine and hyaluronic acid (HA);

vi) a nanoparticle having a core formed from serine and polyethylene glycol monomethyl ether (mPEG); and

v) a nanoparticle having a core formed from serine and poly(L-lysine) (PLL).

In some embodiments, the surface of the nanoparticle is functionalised with at least a carbohydrate.

As used herein, carbohydrate refers to a biomolecule consisting of carbon (C), hydrogen (H) and oxygen (O) atoms. Carbohydrates can have a hydrogen-oxygen atom ratio of 2:1 and thus have an empirical formula Cₘ(H₂O)ₙ (where m may be different from n). This formula holds true for monosaccharides. Some exceptions exist; for example, deoxyribose has the empirical formula C₅H₁₀O₄; these are also included within the scope of carbohydrate. The saccharides are divided into four chemical groups:

monosaccharides, disaccharides, oligosaccharides, and polysaccharides. Examples of carbohydrates include, but is not limited to, fructose, glucose, galactose, xylose, sucrose, lactose, maltose, trehalose, sorbitol, mannitol, maltodextrins, raffinoise, stachyose, fructo-oligosaccharides, amylose, amylopectin, modified starches, glycogen, cellulose, hemicellulose, pectins and hydrocolloids. Stereoisomers such as diastereomers or epimers of carbohydrates are also included within this scope.

In some embodiments, the carbohydrate is mannose. As shown herein, functionalisation with mannose is particularly advantageous for sensing of E. coli. Depending on the bacteria type, other carbohydrates can be used. For example, the carbohydrate can be selected from galactose, xylose, sucrose, lactose, maltose, trehalose, sorbitol, and mannitol.

Mannose is a sugar monomer of the aldohexose series of carbohydrates. It is a C-2 epimer of glucose. Mannose commonly exists as two different-sized rings, the pyranose (six-membered) form and the furanose (five-membered) form. Each ring closure can have either an alpha or beta configuration at the anomeric position, and rapidly undergoes isomerization among these four forms. These configurations, including the linear configurations, are included within the scope of the invention.

In some embodiments, the nanoparticle has a mean diameter of about 1 nm to about 8 nm. In other embodiments, the nanoparticle has a mean diameter of about 1 nm to about 7 nm, 1 nm to about 6 nm, 1 nm to about 5 nm, 1 nm to about 4 nm, or 1 nm to about 3 nm. The mean diameter can be tuned by varying the synthetic conditions. For example, the hydrothermal process can be tuned by varying the temperature, pressure and reaction time.

The photoluminescence stability may be improved by forming a shell around the nanoparticle core. The shell acts to passivate the surface of the core and “blocks” holes that can trap electrons and prevent them from relaxing and releasing luminescence. The shell can comprise a carbohydrate such as mannose. Further, it is possible that a thicker and/or denser shell can be more effective as compared to a thin and/or sparse or patchy shell.

To this end, the shell can comprise other functionalities. For example, PEG moieties can be used to improve the dispersibility of the nanoparticles in an aqueous medium.

In some embodiments, the nanoparticle has a photoluminescence stability of at least 95% after 30 min irradiation. In other embodiments, the photoluminescence stability is at least 92%, at least 90%, at least 85%, at least 80%, at least 75%, or at least 70%.

In some embodiments, the nanoparticle has a zeta potential of more than about +5 mV. In other embodiments, the zeta potential is more than about +7.5 mV, more than about +10 mV, or more than about +15 mV. The zeta potential may be varied by, for example, varying the amount of carbohydrate on the surface of the nanoparticles. For example, by using a lesser amount of carbohydrate during the synthesis, a higher zeta potential for the nanoparticles can be obtained. This can be advantageous when a fast initial interaction with the membrane of the bacterial cell is desired as bacterial cell membrane can have a net negative charge.

In some embodiments, the nanoparticle has a minimum inhibitory concentration (MIC) value against bacteria of more than 150 μg/mL. The minimum inhibitory concentration (MIC) is the lowest concentration of a chemical which prevents visible growth of a bacterium or bacteria. MIC depends several factors, such as the microorganism and the compound (or nanoparticle). In other embodiments, the nanoparticle has a minimum inhibitory concentration (MIC) value against E. coli of more than 150 μg/mL, more than 200 μg/mL, more than 250 μg/mL, more than 300 μg/mL, more than 350 μg/mL, more than 400 μg/mL, or more than 450 μg/mL. In other embodiments, the nanoparticle has a minimum inhibitory concentration (MIC) value against S. aureus of more than 150 μg/mL, more than 200 μg/mL, or more than 250 μg/mL.

Alternatively, the nanoparticle can have a IC50 value. The half maximal inhibitory concentration (IC50) is a measure of the potency of a substance (or nanoparticle) in inhibiting a specific biological or biochemical function. IC50 is a quantitative measure that indicates how much of a particular inhibitory substance is needed to inhibit, in vitro, a given biological process or biological component by 50%. The IC50 value will correspondingly be half that of the MIC value.

Particularly advantageously, the MIC values in the ranges defined above allows the nanoparticles to be suitable for use in bacterial cell assays. To this end, sufficient time is made available for obtaining an output without the bacterial cells being killed prematurely.

The nanoparticle can be maintained as a dispersion or suspension in an appropriate medium in order to form a nanoparticle reagent for use in an assay. Examples of such medium are aqueous medium. For example, water, phosphate buffered saline or other biological buffers can be used for suspending the nanoparticles. Alternatively, the nanoparticles can be freeze-dried as powder for reconstitution when use in an assay.

The term ‘aqueous medium’ used herein refers to a water based solvent or solvent system, and which comprises of mainly water. Such solvents can be either polar or non-polar, and/or either protic or aprotic. Solvent systems refer to combinations of solvents which resulting in a final single phase. Both ‘solvents’ and ‘solvent systems’ can include, and is not limited to, pentane, cyclopentane, hexane, cyclohexane, benzene, toluene, dioxane, chloroform, diethylether, dichloromethane, tetrahydrofuran, ethyl acetate, acetone, dimethylformamide, acetonitrile, dimethyl sulfoxide, nitromethane, propylene carbonate, formic acid, butanol, isopropanol, propanol, ethanol, methanol, acetic acid, ethylene glycol, diethylene glycol or water. Water based solvent or solvent systems can also include dissolved ions, salts and molecules such as amino acids, proteins, sugars and phospholipids. Such salts may be, but not limited to, sodium chloride, potassium chloride, ammonium acetate, magnesium acetate, magnesium chloride, magnesium sulfate, potassium acetate, potassium chloride, sodium acetate, sodium citrate, zinc chloride, HEPES sodium, calcium chloride, ferric nitrate, sodium bicarbonate, potassium phosphate and sodium phosphate. As such, biological fluids, physiological solutions and culture medium also falls within this definition.

The nanoparticle reagent can additional comprise other excipients. For example, an excipient can be added to improve the shelf-life of the nanoparticles. Examples of excipients are, but not limited to, salts such as NaCI, KCI, Na₂HPO_{4,} KH₂PO₄ and small molecules such as TAPS ([tris(hydroxymethyl)methylamino]propanesulfonic acid), Bicine (2-(bis(2-hydroxyethyl)amino)acetic acid), Tris (tris(hydroxymethyl)aminomethane) or (2-amino-2-(hydroxymethyl)propane-1,3-diol), Tricine (N-[tris(hydroxymethyl)methyl]glycine), TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]lethanesulfonic acid), MOPS (3-(N-morpholino)propanesulfonic acid), PIPES (piperazine-N,N′-bis(2-ethanesulfonic acid)), Cacodylate (dimethylarsenic acid), and MES (2-(N-morpholino)ethanesulfonic acid).

In some embodiments, the nanoparticle is characterised by a photoluminescent core having a surface,

wherein the core is an amorphous core formed from threonine and polyethylenimine (PEI);

wherein the weight ratio of threonine and PEI is about 2:1;

wherein the surface is functionalised with mannose; and

wherein the weight ratio of mannose to the core is about 1:1.

The present invention also relates to a method of forming and functionalising a nanoparticle, including the steps of:

a) hydrothermally reacting threonine and PEI to form the nanoparticle having a photoluminescent amorphous core; and

b) reacting at least a carbohydrate with a surface of the nanoparticle core in order to form a surface functionalised nanoparticle with at least a carbohydrate.

The method can be performed as a one pot two step synthesis. For example, the totality of the method can be performed in a hydrothermal reactor. The reactor can be an autoclave, such as a PTFE-lined vessel.

Hydrothermal synthesis or solvothermal synthesis is an approach to synthesising nanoparticles with controlled size and shape under high-temperature and high pressures. The nanoparticles generated are usually monodispersed.

In some embodiments, step (a) is performed at high temperature and pressure. In some embodiments, step (a) is performed at about 150 ° C. to about 250 ° C. In other embodiments, the temperature is about 150 ° C. to about 240 ° C., about 150 ° C. to about 230 ° C., about 150 ° C. to about 220 ° C., about 150 ° C. to about 210 ° C., about 150 ° C. to about 200 ° C., about 160 ° C. to about 200 ° C., about 170 ° C. to about 200 ° C., or about 170 ° C. to about 190 ° C. In other embodiments, the temperature is about 180 ° C.

In some embodiments, step (a) is performed at about 20 psi to about 40 psi. In other embodiments, the pressure is about 22 psi to about 40 psi, about 24 psi to about 40 psi, about 25 psi to about 40 psi, about 25 psi to about 38 psi, about 25 psi to about 36 psi, about 25 psi to about 34 psi, about 25 psi to about 32 psi, or about 25 psi to about 30 psi.

In some embodiments, step (a) is performed for about 14 h to about 24 h, about 16 h to about 24 h, about 18 h to about 24 h, about 18 h to about 22 h, or about 18 h to about 22 h.

In some embodiments, a weight ratio of threonine and PEI is about 2:1. In other embodiments, the weight ratio is about 1.5:1 to about 2.5:1, about 1.6:1 to about 2.5:1, about 1.7:1 to about 2.5:1, about 1.8:1 to about 2.5:1, about 1.8:1 to about 2.4:1, about 1.8:1 to about 2.3:1, about 1.8:1 to about 2.2:1, or about 1.8:1 to about 2.1:1.

In some embodiments, the method further includes a step of filtrating the core from the unreacted threonine and PEI after step (a). The filtration step can be performed by centrifugation, or by passing the reaction mixture through a filter. This has the additional benefit of producing a highly monodispersed nanoparticle distribution.

In some embodiments, step (b) is performed at about 60 ° C. for about 48 h. In other embodiments, the temperature is about 70 ° C., about 80 ° C., about 90 ° C., about 100 ° C., or about 110 ° C. In other embodiments, the reaction time is about 10 h, about 15 h, about 20 h, about 25 h, about 30 h, about 40 h, about 50 h, about 60 h, or 80 h.

In some embodiments, step (b) is performed at atmospheric pressure. In this regard, the pressure is about 14 psi to about 16 psi, or about 15 psi.

In some embodiments, a weight ratio of carbohydrate to the core of the nanoparticle is about 1:1. In other embodiments, the weight ratio is about 0.8:1 to about 1.2:1, about 0.9:1 to about 1.2:1, about 0.8:1 to about 1.1:1, or about 0.9:1 to about 1.1:1. In other embodiments, the weight ratio is about 1:0.8 to about 1:1.2, about 1:0.9 to about 1:1.2, about 1:0.8 to about 1:1.1, or about 1:0.9 to about 1:1.1.

In some embodiments, the method further includes a step of purifying the nanoparticle after step (b). The purification step can be performed by centrifugation, or by passing the reaction mixture through a filter.

In some embodiments, the method is performed in a medium selected from an aqueous medium. For example, the medium is a solvent selected from water.

When the presently disclosed nanoparticles (and/or nanoparticle reagent) are combined with antibody conjugated silica coated magnetic particles, a bacteria sensing assay with 3 steps involving magnetic capture, magnetic concentration and fluorescence labelling is able to achieve a quantification detection of E.coli down to about 10⁴ CFU/mL.

Accordingly, the present invention also relates to a photoluminescence assay for quantifying a sample comprising bacterial cells, including the steps of:

a) incubating the sample with a silica coated magnetic particle for allowing the silica coated magnetic particle to contact the bacterial cells;

b) exposing the bacterial cells contacted with the silica coated magnetic particle to an external magnetic field to form a first magnetic pellet and a first supernatant;

c) separating the first magnetic pellet from the first supernatant;

d) incubating the first magnetic pellet with a nanoparticle for allowing the nanoparticle to contact the bacterial cells, the nanoparticle is characterised by a photoluminescent amorphous core having a surface, wherein the core is formed from threonine and polyethylenimine (PEI), and wherein the surface is functionalised with at least a carbohydrate;

e) exposing the bacterial cells contacted with the silica coated magnetic particle and the nanoparticle to an external magnetic field to form a second magnetic pellet and a second supernatant; and

f) quantifying the emittable photoluminescence from the second magnetic pellet.

The assay method can also be performed using the nanoparticle reagent as disclosed herein.

In some embodiments, incubation step (a) is performed for at least 10 min. In other embodiments, the time is at least 15 min, at least 20 min, at least 25 min, at least 30 min, at least 40 min, or 60 min.

As used herein, magnetic particles refer to nanoparticle that can be manipulated using magnetic fields. In some embodiments, the magnetic particle comprises a transition metal selected from iron, cobalt and nickel. In other embodiments, the magnetic particle is an iron oxide particle. In other embodiments, the magnetic particle is Fe₃O₄ or Fe₂O₃.

In some embodiments, the silica coated magnetic particle has a mean particle size of about 15 nm to about 40 nm. In other embodiments, the mean particle size is about 20 nm to about 40 nm, about 30 nm to about 40 nm, about 30 nm to about 50 nm, about 40 nm to about 60 nm, about 50 nm to about 70 nm, or about 60 nm to about 80 nm.

In some embodiments, the silica coated magnetic particle has a silica shell thickness of at least about 5 nm. In other embodiments, the silica shell thickness is at least about 6 nm, about 7 nm, about 8 nm, about 9 nm, about 10 nm, or about 15 nm.

In some embodiments, the silica coated magnetic particle has a magnetic particle core mean particle size of about 10 nm. In other embodiments, the particle core is about 12 nm, about 15 nm, about 20 nm, about 30 nm, about 40 nm, or about 50 nm.

In some embodiments, the silica coated magnetic particle has a saturation magnetization of more than about 40 emu/g. Saturation magnetization is the state when an increase in applied external magnetic field H cannot further increase the magnetization of a material, so the total magnetic flux density B more or less levels off. Saturation is most clearly seen in a magnetization curve (also called BH curve or hysteresis curve) of the material, as a bending or plateau to the right of the curve. As the H field increases, the B field approaches a maximum value asymptotically, the saturation level for the material. In other embodiments, the saturation magnetization is more than about 50 emu/g, about 60 emu/g, about 70 emu/g, about 80 emu/g, about 90 emu/g, or about 100 emu/g.

In some embodiments, the silica coated magnetic particle has negligible remanence. Remanence or remanent magnetization or residual magnetism is the magnetization left behind in a ferromagnetic material (such as iron) after an external magnetic field is removed.

In some embodiments, the silica coated magnetic particle has a zeta potential of about −5 mV to about +5 mV. In other embodiments, the silica coated magnetic particle has a zeta potential of about −10 mV to about +10 mV. In other embodiments, the silica coated magnetic particle has a zeta potential of about −20 mV to about +20 mV.

In some embodiments, the silica coated magnetic particle is at least partially functionalised with an amino moiety. In other embodiments, the silica coated magnetic particle is at least 10% functionalised with an amino moiety, at least 20%, at least 30%, at least 40%, at least 50%, at least 60%, at least 70%, at least 80%, or at least 90%. In certain embodiments, the silica coated magnetic particle has a surface which is about 40% functionalised with an amino moiety. In other embodiments, the amino moiety is selected from amine, methylamine, ethylamine, propylamine, dimethylamine, or diethylamine.

To partially functionalise the magnetic particles, a ratio of silica precursors can be used. In some embodiments, the percent ratio (based on moles) of tetraethyl orthosilicate (TEOS) to (3-aminopropyl)triethoxysilane (APTES) is about 85:15 to about 50:50. In other embodiments, the ratio is about 80:20 to about 50:50, about 70:30 to about 50:50, or about 60:40 to about 50:50. In certain embodiments, the percent ratio (based on moles) of tetraethyl orthosilicate (TEOS) to (3-aminopropyl)triethoxysilane (APTES) is about 60:40.

Advantageously, by controlling the amount of amine functionalisation on the magnetic particles, it is possible to control the density of antibody conjugation on the magnetic particle. This allows the active site on the antibody to be properly exposed for interaction with the bacterial cell.

In some embodiments, the silica coated magnetic particle is conjugated to an antibody for targeting bacterial cells. The antibody can be conjugated to the silica coated magnetic particle by any known conjugation means. For example, carbodiimide crosslinking chemistry such as 1-ethyl-3-(-3-dimethylaminopropyl) carbodiimide (EDC) can be used to derivatize the antibody such that it can covalently bond with the amino moiety on the silica coated magnetic particle to form an amide bond.

The antibody can be selected to target specific bacterial cells. In some embodiments, the antibody is a monoclonal or polyclonal antibody. In other embodiments, the antibody specifically targets E.coli.

In some embodiments, the sample is incubated with silica coated magnetic particle at a concentration of about 1 mg/mL. In other embodiments, the concentration is about 0.5 mg/mL, about 1.5 mg/mL, or about 2 mg/mL.

The step of exposing the bacterial cells to an external magnetic field (step b) allows for the separation of the bacterial cells from the other impurities and excess reagents in the supernatant. Advantageously, this step does not adversely affect the bacterial cells and a greater amount of bacterial cells can be isolated for better accuracy. In contrast, methods such as centrifugation can damage or rupture the cells and a lower total separation is achieved. In some embodiments, the bacterial cells are exposed for more than about 10 min, about 20 min, about 30 min, about 40 min, about 50 min, or about 60 min.

The first magnetic pellet can be separated from the first supernatant by decanting the supernatant. Alternatively, the magnetic pellet can be filtered to separate it from the supernatant.

The first magnetic pellet can be resuspended in an appropriate solvent or medium. The resuspended bacterial cells comprises silica coated magnetic particles on the bacterial cell membrane. When incubated with the nanoparticles as disclosed herein, the nanoparticles can contact and/or adhere to the bacterial cells, thus “marking” the bacterial cells.

In some embodiments, the first magnetic pellet (or its resuspended form thereof) is incubated with the nanoparticles for more than about 5 min, about 10 min, about 20 min, about 30 min, about 40 min, about 50 min, or about 60 min.

In some embodiments, the first magnetic pellet is incubated with nanoparticles at a concentration of about 1 mg/mL. In other embodiments, the concentration is about 0.5 mg/mL, about 1.5 mg/mL, or about 2 mg/mL.

After incubation with nanoparticles, the step of exposing the bacterial cells to an external magnetic field (step e) allows for the separation of the bacterial cells from the other impurities and excess reagents in the supernatant. In some embodiments, the bacterial cells are exposed for more than about 10 min, about 20 min, about 30 min, about 40 min, about 50 min, or about 60 min.

In some embodiments, the assay further includes a step of resuspending the second magnetic pellet in a solvent or medium after step (e). Resuspending the second magnetic pellet can allow for better accuracy in quantification as self-quenching of the photoluminescence (fluorescence) is minimised.

The emittable photoluminescence from the second magnetic pellet can thus be quantified. As is clear from the discussion above, the photoluminescence is emittable from the nanoparticles, which are correspondingly contacted with the bacterial cells. It would be clear to the skilled person that a number of nanoparticles can be contacted with a bacterial cell. Accordingly, the amount of bacterial cells can be quantified by comparing with a calibration plot.

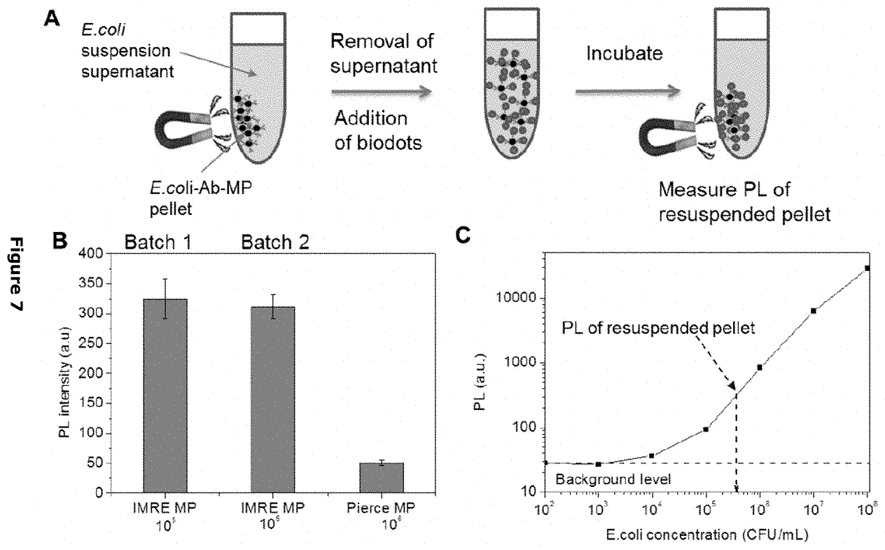

In some embodiments, the assay further includes a step of comparing the emitted photoluminescence with a calibration plot for determining the concentration of bacterial cells in the sample after step (f). An example of a calibration plot is shown in FIG. 7C.

In some embodiments, a weight ratio of nanoparticles to magnetic particles is about 1:1.

Advantageously, by keeping within the above weight ratio, wastage of reagents can be minimised. Advantageously, the above assay method can be performed at ambient temperature (about 15 ° C. to about 28 ° C.) and pressure.

In some embodiments, the photoluminescence assay for quantifying a sample comprising E. coli bacterial cells. In other embodiments, the assay is for quantifying Gram-positive bacterial cells selected from S. saprophyticus, S. bovis, C. urealyticum, S. aureus, B. cereus, S. pneumonia, A. adiacens, S. mitis group, S. agalactiae, S.

lugdunensis, C. jelkenium, Lactobacillus sp., C. septicum, Veillonella, Eubacterium, Clostridium sp., C. difficile, C. perfringens, Listeria monocytogenes, Erysipelothrix rhusiopathiae, Arcanobacterium bemolyticum, Bacillus megaterium, Bacillus subtilis, Brevibacterium linens, Corynebacterium glutamicum, Mycobacterium smegmatis, Rhodococcus erythropolis, and Streptomyces lividans. In other embodiments, the assay is for quantifying Gram-negative bacterial cells selected from Proteus spp., Escherichia coli, Morganella, N. gonorrhoea, Moraxella catarrhalis, N. meningitides, Klebsiella pneumonia, Aeromonas hydrophila, Providencia, Enterobacter cloacae, Cariobacter hominis, H. influenza, Alkaligenes, Burkholderia cepacia, H. parainfluenzae, Pasturella multocida, Kingella kingae, Brucella sp., C. jejuni, Haemophilus aphrophilus,

Pseudomonas, and Stenotrophomonas maltophilia, Agrobacterium tumefaciens,

Azospirillum brasilense, Bordetella avium, Brevundimonas diminuta, Burkholderia cepacia, Burkholderia gladioli, Burholderia vietnamensis, Chromobacterium violaceum, Citrobacter freundii, Enterbacter aerogenes, Erwinia amylovora, Erwinia carotovora, Proteus vulgaris, Rhizobium etli, Salmonella enteritidis, Serratia entomophila, Shigella flexneri, Sphingomonas wittichii, Variovorax paradoxus, Vibrio harveyi, Xanthomonas, and Yersinia enterocolitica. In some embodiments, the bacterial cell is foodborne pathogen selected from Clostridium botulinum, Campylobacter jejuni, Escherichia coli, Listeria monocytogenes, Salmonella, Shiga toxin-producing E. coli such as verocytotoxic E. coli (VTEC) or enterohemorrhagic E. coli (EHEC), Shigella, Staphylococcus aureus, and Vibrio parahaemolyticus. The skilled person would understand that to target other bacterial cells, the antibody on the magnetic particle can be changed. Other carbohydrates may also be used to improve the detection limit of the assay for other bacterial cells.

Accordingly, in some embodiments, the photoluminescence assay for quantifying a sample comprising E. coli bacterial cells, includes the steps of:

a) incubating the sample with a silica coated magnetic particle at a concentration of about 1 mg/mL for allowing the silica coated magnetic particle to contact the bacterial cells;

b) exposing the bacterial cells contacted with the silica coated magnetic particle to an external magnetic field to form a first magnetic pellet and a first supernatant;

c) separating the first magnetic pellet from the first supernatant;

d) incubating the first magnetic pellet with a nanoparticle at a concentration of about 1 mg/mL for allowing the nanoparticle to contact the bacterial cells, the nanoparticle is characterised by a photoluminescent amorphous core having a surface, wherein the core is formed from threonine and polyethylenimine (PEI), and wherein the surface is functionalised with mannose;

e) exposing the bacterial cells contacted with the silica coated magnetic particle and the nanoparticle to an external magnetic field to form a second magnetic pellet and a second supernatant; and

f) quantifying the emittable photoluminescence from the second magnetic pellet.

Advantageously, the combination of silica coated magnetic particles and nanoparticles of the present invention provides a synergistic effect for detecting bacterial cells. Bacterial cells can be isolated using the silica coated magnetic particles, which are subsequently interacted with the nanoparticles for quantification. It is believed that the interaction of bacterial cells with silica coated magnetic particles increases the susceptibility of the bacterial cells with nanoparticles, thus increasing the emittable photoluminescence. The photoluminescence from the assay is also higher because the magnetic particles not only specifically target the bacteria, but also help to concentrate the bacteria thus increasing the overall photoluminescence after resuspension which improves the assay sensitivity.

In some embodiments, the assay has a detection limit of at least 1×10⁴ CFU/mL of bacterial cells. In other embodiments, the detection limit is at least 5×10⁴ CFU/mL, at least 1×10⁵ CFU/mL, at least 5×10⁵ CFU/mL, at least 1×10⁶ CFU/mL, or at least 5×10⁶ CFU/mL.

Advantages of invention are as follows:

a) A multifunctional TPM biodot with desirable surface functional groups to interact with bacteria with high affinity selective targeting of specific bacteria species

b) A photoluminescent biodot with enhanced brightness and excellent photostability that show 98.7% PL remaining after 30 min UV irradiation stable fluorescence signal

c) A ultrasmall (1-5 nm) biodot with slightly positive zeta potential (7 mV) for fast initial interaction with bacterial membrane

d) Fast detection

e) A multifunctional biodot that exhibit effective bacteria inhibition capability

f) A non-quenching antibody conjugated silica coated magnetic particles for efficient bacteria separation that achieves ˜100% removal of E.coli at 10⁵ CFU initial bacteria amount

g) A proof-of-concept fluorescence based method for quantitative E.coli detection using the functionalized magnetic particles and biodotswith a detection limit of 10⁴ CFU/mL and assay time of ˜1 h.

The present invention has the following applications:

a) Quantitation of bacteria count in food and beverages

b) Bacteria sensing in aquatic systems

c) Air microbial monitoring

d) Bacteria detection in pharmaceutical and cosmetic industries

e) Pathogenic bacteria detection in clinical samples

The present invention can be used in processing, preparing and manufacturing foods safely. The importance of diligent cleaning, disinfecting and sanitizing in these processes cannot be undermined. To this end, the nanoparticles and/or assay of the present invention can, for example, be used to identify, eliminate or control food-borne pathogens.

Further, large scale production of food is an ongoing challenge in that many variable factors come into play for ensuring quality and food safety. Of note, contamination due to bacteria is of importance as a slight or minor contamination can void the whole batch, resulting in large wastage and monetary losses. To this end, the nanoparticles and/or assay of the present invention can, for example, be used to troubleshoot for entry or contamination points.

The present invention can also be used for monitoring pathogenic bacteria in aquacultures and detecting microbial contamination in cosmetic and pharmaceutical products. In this regard, a sample can be obtained and through conjugation with the magnetic particles, can be concentration for fluorescence quantification.

### EXAMPLES

### 1. Molecular Engineering of Biodots with Enhanced Labelling Intensity and Specific Affinity Toward E.Coli

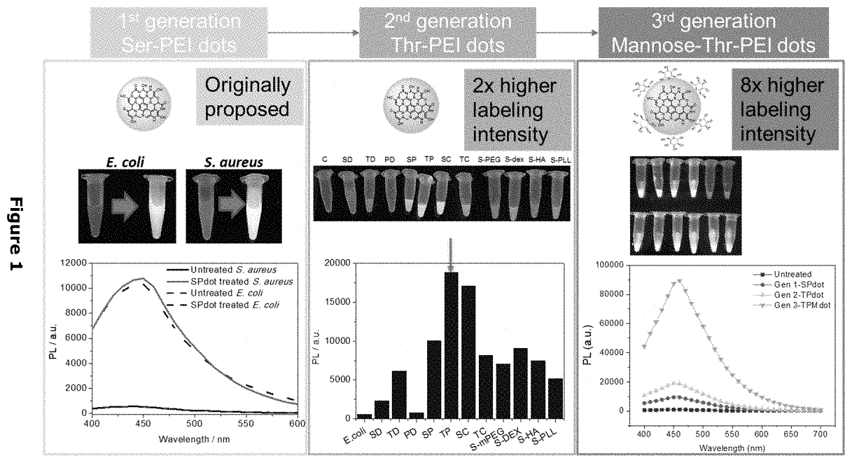

In this embodiment, the inventors have molecularly engineered the biodot (or nanoparticle) surface to exhibit high brightness by selecting the optimal amino acid and polymer pair (i.e. threonine and PEI) as well as moieties with high affinity to a specific bacteria strain (i.e. mannose). Thr-PEI-mannose (TPM) dots were synthesised which have shown 8 times higher labeling intensity for E.coli as compared to the 1st generation Ser-PEI (SP) dots and 4 times higher than the 2nd generation Thr-PEI (TP) dot as shown in FIG. 1. It is expected that the improvement in labelling intensity will lead to the lowering of detection limits.

The synthesis route for TPMdots follows a two-step hydrothermal process as shown FIG. 2A. The amino acid, threonine is first mixed with branched PEI at high temperature and pressure to obtain TP dots. After ultrafiltration, the purified TPdots (<3 kD) are then mixed with mannose for a second step heating process (60° C., 48 h) to obtain the final TPMdots.

The properties of TPMdots as compared to the SPdots or TPdots were investigated. The absorbance spectra in FIG. 2A indicates that TPMdots possess both absorbance signature for TPdot and mannose moieties. It is observed from FIG. 2C that both TPMdots and TPdots exhibit two times higher intrinsic photoluminescence than SPdot, suggesting the potential to achieve better detection sensitivity using TPMdots. In addition, the presence of mannose signature absorption peak in TPMdots hints towards its specificity to E.coli, which potentially improves the TPMdot—E.coli interaction, and account for the eight times higher PL labeling intensity for E.coli. The ultra-small TPMdots (1-5 nm) may allow its uptake into the bacterial cells (FIG. 2D). In addition, TPMdots show slightly positive zeta potential (˜+7 mV) that can possibly enhance initial interactions of TPMdot with bacterial membrane for rapid analysis (FIG. 2E). It is also noteworthy to mention that TPMdots show 98.7% PL remaining after 30 min UV irradiation, thereby indicating that it is sufficiently photo-stable for the assay which will be scanned very quickly within minutes (FIG. 2F).

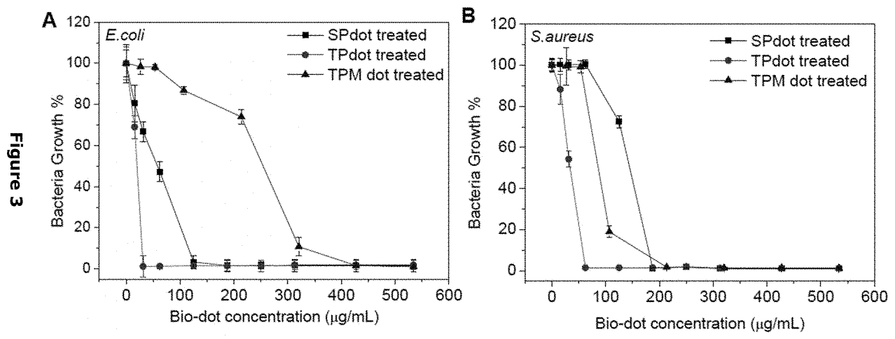

The bacterial inhibition properties of all three generations of biodots were also investigated (FIG. 3). TP dots show most effective bacterial inhibition with the lowest MIC values for both gram-negative E.coli (31 μg/mL) and gram-positive S. aureus (62 μg/mL) after 24 h incubation. In summary, the order of antimicrobial efficiency is TP>SP>TPMdot for E.coli and TP>TPM>SPdot for S.aureus.

### 2. Antibody Conjugated Magnetic Particle for Specific Bacteria Capture

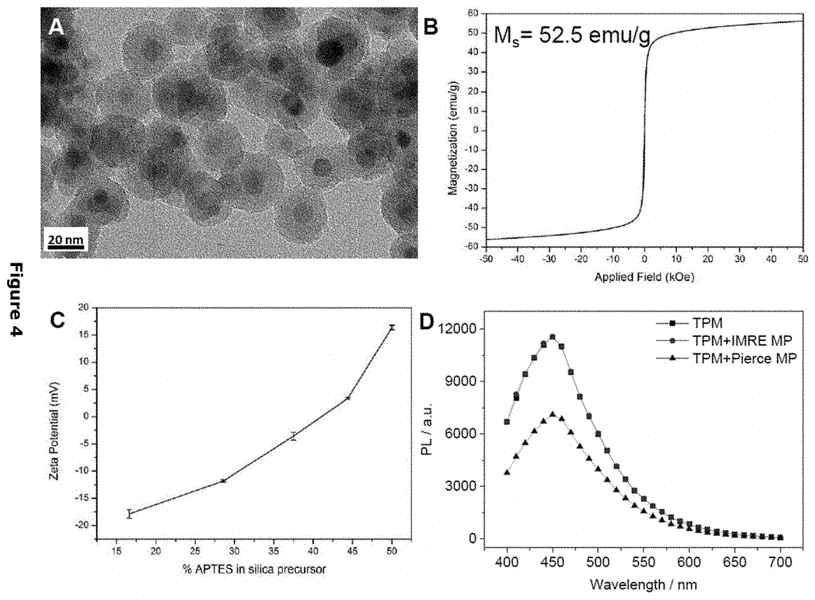

Superparamagnetic iron oxide nanoparticles about 10 nm in size are first synthesized. These iron oxide nanoparticles are subsequently encapsulated with silica through the hydrolysis and condensation of tetraethyl orthosilicate (TEOS) and (3-aminopropyl)triethoxysilane (APTES) inside the micelles of a water-in-oil microemulsion. The TEM image (FIG. 4A) showed that majority of the resulting magnetic particles (MP) had an iron oxide core-silica shell configuration. In addition, the silica coated MP exhibited a high saturation magnetization of 52.5 emu/g and negligible remanence (FIG. 4B); this indicates that the silica encapsulation process has not affected the superparamagnetic behaviour.

Employing the silica coated MP in the fluorescent assay offers two critical advantages. Firstly, due to the presence of a silica coating, it would minimize the fluorescence quenching of the biodots that were used to stain the bacteria (FIG. 4D). Correspondingly, this would improve on the sensitivity of the fluorescent assay.

Secondly, in lieu of the versatile silane chemistry available, it would also facilitate the subsequent surface modification (e.g. amine functionalization) of the silica coating. The control of these two properties allows for the suppression of fluorescence quenching as well as for achieving a delicate balance between effective antibody functionalization and surface charge control to improve bacteria capture and minimize non-specific attachment. The zeta potential of the MP could also be specifically tuned by varying the molar TEOS/APTES ratio (FIG. 4C), thereby allowing for surface charge optimization to minimize non-specific bacteria interactions. Herein, it is noteworthy that for MP with bare silica coating (i.e. no amine functionalization), the determined surface charge is -39.0 mV; but the introduction of amino groups (pKa=9.0) onto the silica surface would result in a positive shift in the zeta potential measurements.

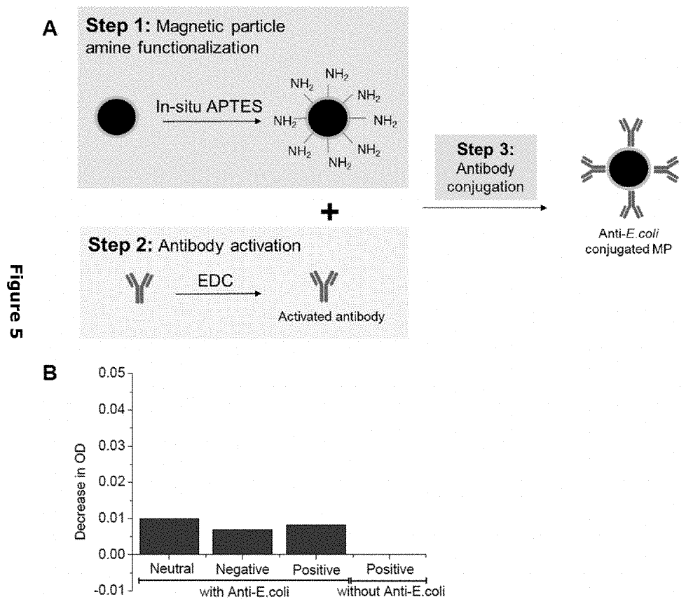

EDC was used to activate the antibodies for direct conjugation to in-situ APTES functionalized magnetic particles (FIG. 5A). Silica coated magnetic particles with different zeta potentials (−15, −5 and +14 mV) were used to check the effect of zeta potential on conjugation effectiveness. Based on OD readings, the bacteria capture efficiency of conjugated magnetic particles with a near neutral zeta potential (−5 mV) was slightly more effective compared to a negative or positive zeta potential (FIG. 5B).

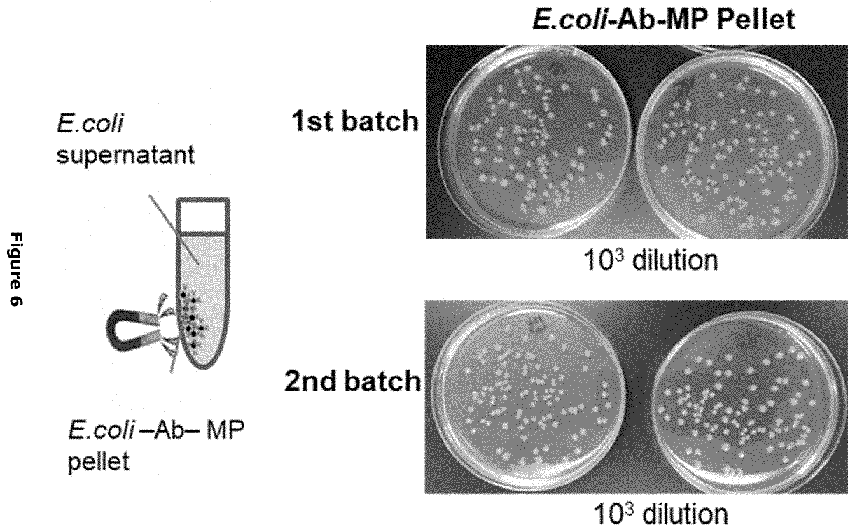

The amount of bacteria captured by the anti-E.coli conjugated magnetic particles were further verified by plate counting. 1 mL of E.coli suspension containing 10⁵ CFU was incubated with 1 mg of magnetic particles for 0.5 h. The magnetic pellet was removed from the bacteria suspension and washed twice with PBS. It was then reconstituted in 1 mL of PBS. Colony counting shows that 1 mg of conjugated magnetic particles (carbodiimide method) is able to capture an average of 1.10×10⁵ CFU/mL of E.coli (FIG. 6). On the other hand, control magnetic particles with no antibody conjugation can only capture about 2.0×10² CFU/mL of E.coli. Direct conjugation of antibodies to magnetic particles using carbodiimide chemistry is effective. The results showed that the conjugated magnetic particles obtained from the above presented conjugation strategies are highly specific to E.coli and reproducible.

### 3. Fluorescence Assay for E.Coli Quantification

To carry out fluorescence based detection of E.coli, the anti-E.coli antibody functionalized magnetic particles were first incubated with E.coli samples for 30 min (FIG. 7A). E.coli were specifically captured onto the magnetic particles and TPMdots were then added to label the E.coli captured. Finally, the PL of the resuspended pellet containing TPMdot stained E.coli captured on MP were measured to quantify the E.coli concentration.

As shown in FIG. 7B, the PL signal from 10⁵ CFU E.coli captured on IMRE MP (antibody conjugated silica coated MP) is much higher than the PL signal from 10⁶ E.coli captured on a commercially available MP which are not silica coated (Pierce), thus proving that the unique properties (non-quenching) of IMRE MP is critical to the fluorescence assay. Further, multiple TPMdots can label a single bacteria and hence improve the detection sensitivity. In addition, the PL intensity numbers obtained from two independent experiment are reproducible. When the PL intensity value is compared to the calibration curve (FIG. 7C), the fluorescence intensity value also corresponds to 10⁵ CFU/mL E.coli, thereby allowing specific bacterial quantification. According to the calibration curve, as low as 10⁴ CFU/mL E.coli can be detected with the current assay.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A nanoparticle which is characterised by a photoluminescent core having a surface,
wherein the core is an amorphous core formed from threonine and polyethylenimine (PEI); and
wherein the surface is functionalised with at least a carbohydrate, wherein the photoluminescence from the core is emittable within the range of about 400 nm to about 650 nm.

2. (canceled)

3. The nanoparticle according to claim 1, wherein the emittable photoluminescence is at least 1.5 times a comparator nanoparticle which does not comprise both threonine and PEI, wherein the comparator is selected from the group consisting of:
i) a nanoparticle having a core formed from serine or threonine or PEI;
ii) a nanoparticle having a core formed from serine and PEI;
iii) a nanoparticle having a core formed from threonine and chitosan;
iv) a nanoparticle having a core formed from serine and dextran (DEX);
v) a nanoparticle having a core formed from serine and hyaluronic acid (HA);
vi) a nanoparticle having a core formed from serine and polyethylene glycol monomethyl ether (mPEG); and
vii) a nanoparticle having a core formed from serine and poly(L-lysine) (PLL).

4. (canceled)

5. The nanoparticle according to claim 1, wherein the PEI is a branched PEI, and/or wherein the core has a molecular weight of less than 3 kD.

6. (canceled)

7. The nanoparticle according to claim 1, wherein the carbohydrate is mannose.

8. The nanoparticle according to claim 1, having a mean diameter of about 1 nm to about 8 nm.

9. The nanoparticle according to claim 1, having a photoluminescence stability of at least 95% after 30 min irradiation, wherein the photoluminescence is fluorescence.

10. (canceled)

11. The nanoparticle according to claim 1, having a zeta potential of more than about +5 mV, and/or having a minimum inhibitory concentration (MIC) value against bacteria of more than 150 μg/mL.

12. (canceled)

13. A method of forming and functionalising a nanoparticle, including the steps of:
a) hydrothermally reacting threonine and PEI to form the nanoparticle having a photoluminescent amorphous core; and
b) reacting at least a carbohydrate with a surface of the nanoparticle core in order to form a surface functionalised nanoparticle with at least a carbohydrate.

14. (canceled)

15. The method according to claim 13, wherein step (b) is performed at about 60 ° C. for about 48 h.

16. The method according to claim 13, further including a step of filtrating the core from the unreacted threonine and PEI after step (a).

17. The method according to claim 13, further including a step of purifying the nanoparticle after step (b).

18. A photoluminescence assay for quantifying a sample comprising bacterial cells, including the steps of:
a) incubating the sample with a silica coated magnetic particle for allowing the silica coated magnetic particle to contact the bacterial cells;
b) exposing the bacterial cells contacted with the silica coated magnetic particle to an external magnetic field to form a first magnetic pellet and a first supernatant;
c) separating the first magnetic pellet from the first supernatant;
d) incubating the first magnetic pellet with a nanoparticle for allowing the nanoparticle to contact the bacterial cells, the nanoparticle is characterised by a photoluminescent amorphous core having a surface, wherein the core comprises threonine and polyethylenimine (PEI), and wherein the surface is functionalised with at least a carbohydrate;
e) exposing the bacterial cells contacted with the silica coated magnetic particle and the nanoparticle to an external magnetic field to form a second magnetic pellet and a second supernatant; and
f) quantifying the emittable photoluminescence from the second magnetic pellet.

19. The assay according to claim 18, wherein step (a) is performed for at least 10 min.

20. The assay according to claim 18, wherein the silica coated magnetic particle has a mean particle size of about 15 nm to about 40 nm.

21. The assay according to claim 18, wherein the silica coated magnetic particle has a silica shell thickness of at least about 5 nm, wherein the silica coated magnetic particle has a magnetic particle core mean particle size of about 10 nm.

22. (canceled)

23. The assay according to claim 18, wherein the silica coated magnetic particle has a saturation magnetization of more than about 40 emu/g, and/or wherein the silica coated magnetic particle has negligible remanence, and/or wherein the silica coated magnetic particle has a zeta potential of about −5 mV to about +5 mV.

24. (canceled)

25. (canceled)

26. The assay according to claim 18, wherein the silica coated magnetic particle is at least partially functionalised with an amino moiety, and/or wherein the silica coated magnetic particle is conjugated to an antibody for targeting bacterial cells.

27. (canceled)

28. The assay according to claim 18, further including a step of resuspending the second magnetic pellet in a solvent after step (e).

29. The assay according to claim 18, further including a step of comparing the emitted photoluminescence with a calibration plot for determining the concentration of bacterial cells in the sample after step (f).

30. The assay according to claim 18, having a detection limit of at least 104 CFU/mL of bacterial cells.
